(19) United States
(12) Patent Application Publication
(10) Pub. No.: US 2014/0309515 A1
(43) Pub. Date: Oct. 16, 2014

(54) PHOTOACOUSTIC WAVE MEASUREMENT DEVICE, METHOD, AND RECORDING MEDIUM

(71) Applicant: ADVANTEST CORPORATION, Tokyo (JP)

(72) Inventor: Taiichiro IDA, Gunma (JP)

(21) Appl. No.: 14/245,256

(22) Filed: Apr. 4, 2014

(30) Foreign Application Priority Data
Apr. 15, 2013 (JP) 2013-84519

(51) Int. Cl. A61B 5/00

(52) U.S. Cl. CPC A61B 5/0095; A61B 5/489
USPC 600/407

(57) ABSTRACT

A photoacoustic wave measurement device receives electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal. The photoacoustic wave measurement device includes a time deviation determination unit that determines whether time deviations among the electric signals output from the respective photoacoustic wave detection units are in a predetermined range; and a position measurement unit that measures a position of a photoacoustic wave generation part of the measurement object which generates the photoacoustic wave if the time deviation determination unit determines that the time deviations among the electric signals are in the predetermined range.

### BACKGROUND ART

1. Field of the Invention

The present invention relates to a position measurement of a target by means of a photoacoustic sensor.

2. Related Art

It is conventionally known to measure a measurement object by detecting photoacoustic waves by using at least two photoacoustic sensors (refer to Patent Document 1). The photoacoustic sensor radiates light upon the measurement object. Then, the light is absorbed by a target in the measurement object. As a result, the target (photoacoustic wave generation part) generates photoacoustic waves. The photoacoustic sensor detects the photoacoustic wave. If the photoacoustic sensor is positioned directly above the target, the photoacoustic sensor can detect the photoacoustic wave. As a result, the position of the target can be measured.

### PRIOR ART DOCUMENTS

(Patent Document 1) Japanese Patent Application Laid-Open No. 2004-201749
(Patent Document 2) Japanese Patent Application Laid-Open No. 2009-39266
(Patent Document 3) Japanese Patent Application Laid-Open No. 2010-63617

### SUMMARY OF THE INVENTION

However, the photoacoustic wave generated by the target (photoacoustic wave generation part) transmits not only direction directly upward above the target, but also transmits obliquely upward above the target. As a result, if the photoacoustic sensor detects the photoacoustic wave transmitting obliquely upward above the target, the target does not exist directly below the target. In this case, an error is generated in the measurement of the position of the target.

It is therefore an object of the present invention to precisely measure the position of the photoacoustic wave generation part by the photoacoustic wave measurement instrument.

According to the present invention, a photoacoustic wave measurement device for receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, includes: a time deviation determination unit that determines whether time deviations among the electric signals output from the respective photoacoustic wave detection units are in a predetermined range; and a position measurement unit that measures a position of a photoacoustic wave generation part of the measurement object which generates the photoacoustic wave if the time deviation determination unit determines that the time deviations among the electric signals are in the predetermined range.

According to the thus constructed photoacoustic wave measurement device, a photoacoustic wave measurement device for receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, can be provided. A time deviation determination unit determines whether time deviations among the electric signals output from the respective photoacoustic wave detection units are in a predetermined range. A position measurement unit measures a position of a photoacoustic wave generation part of the measurement object which generates the photoacoustic wave if the time deviation determination unit determines that the time deviations among the electric signals are in the predetermined range.

According to the photoacoustic wave measurement device of the present invention, the position measurement unit may measure the position of the photoacoustic wave generation part while it is assumed that the photoacoustic wave generation part exists on an extension line of the light output unit.

According to the photoacoustic wave measurement device of the present invention, the predetermined range may be equal to or more than 0, and equal to or less than a predetermined time threshold.

According to the photoacoustic wave measurement device of the present invention, the predetermined range may include a time deviation of the electric signal output from each of the photoacoustic wave detection units while it is assumed that the photoacoustic wave generation part exists on an extension line of the light output unit.

According to the photoacoustic wave measurement device of the present invention, the predetermined range may not include 0.

According to the present invention, the photoacoustic wave measurement device includes a magnitude determination unit that determines a magnitude relationship between a magnitude of the electric signal output from each of the photoacoustic wave detection units and a predetermined magnitude threshold, wherein the position measurement unit measures the position of the photoacoustic wave generation part of the measurement object which generates the photoacoustic wave if the magnitude determination unit determines that the magnitudes of the electric signals are more than the magnitude threshold, and the time deviation determination unit determines that the time deviations among the electric signals are in the predetermined range.

According to the present invention, a photoacoustic wave measurement method of measuring a photoacoustic wave by receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, includes: a time deviation determination step that determines whether time deviations among the electric signals output from the respective photoacoustic wave detection units are in a predetermined range; and a position measurement step that measures a position of a photoacoustic wave generation part of the measurement object which generates the photoacoustic wave if the time deviation determination step determines that the time deviations among the electric signals are in the predetermined range.

The present invention is a computer-readable medium having a program of instructions for execution by a computer to perform a photoacoustic wave measurement process of measuring a photoacoustic wave by receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, the process including: a time deviation determination step that determines whether time deviations among the electric signals output from the respective photoacoustic wave detection units are in a predetermined range; and a position measurement step that measures a position of a photoacoustic wave generation part of the measurement object which generates the photoacoustic wave if the time deviation determination step determines that the time deviations among the electric signals are in the predetermined range.

### BRIEF DESCRIPTION OF THE DRAWINGS

FIG. 1 is a cross sectional view of a photoacoustic wave measurement instrument 1 according to a first embodiment of the present invention;

FIG. 2 is a plan view of the photoacoustic wave measurement instrument 1 according to the first embodiment of the present invention;

FIG. 3 is a functional block diagram showing a configuration of the photoacoustic wave measurement device 40 according to the first embodiment of the present invention;

FIG. 4 includes charts showing the relationships between the time and the voltage, which are the measurement results by the electric signal measurement units 41 and 42 of the photoacoustic wave measurement device 40 according to the first embodiment, and shows the measurement result of the electric signals obtained from the photoacoustic wave measurement instrument 1 in FIG. 1(a) (FIG. 4(a)), the measurement result of the electric signals obtained from the photoacoustic wave measurement instrument 1 in FIG. 1(b) (FIG. 4(b)), and the measurement result of the electric signals obtained from the photoacoustic wave measurement instrument 1 in FIG. 1(c) (FIG. 4(c));

FIG. 5 includes a cross sectional view (FIG. 5(a)) and a plan view (FIG. 5(b)) of the photoacoustic wave measurement instrument 1 according to the second embodiment of the present invention;

FIG. 6 is a cross sectional view of the photoacoustic wave measurement instrument 1 while the photoacoustic wave measurement instrument 1 according to the second embodiment of the present invention is scanned along the measurement object 2;

FIG. 7 includes charts showing relationships between the time and the voltage, which are the measurement results by the electric signal measurement units 41 and 42 of the photoacoustic wave measurement device 40 according to the second embodiment, and shows the measurement result of the electric signals obtained from the photoacoustic wave measurement instrument 1 in FIG. 6(a) (FIG. 7(a)), the measurement result of the electric signals obtained from the photoacoustic wave measurement instrument 1 in FIG. 6(b) (FIG. 7(b)), and the measurement result of the electric signals obtained from the photoacoustic wave measurement instrument 1 in FIG. 6(c) (FIG. 7(c));

FIG. 8 includes a plan view of the photoacoustic wave measurement instrument 1 including three photoacoustic wave detection units (FIG. 8(a)), and a plan view of the photoacoustic wave measurement instrument 1 including four photoacoustic wave detection units (FIG. 8(b)); and

FIG. 9 is a functional block diagram showing a configuration of the photoacoustic wave measurement device 40 according to the variation of the first embodiment of the present invention.

### MODE FOR CARRYING OUT THE INVENTION

A description will now be given of an embodiment of the present invention referring to drawings.

### First Embodiment

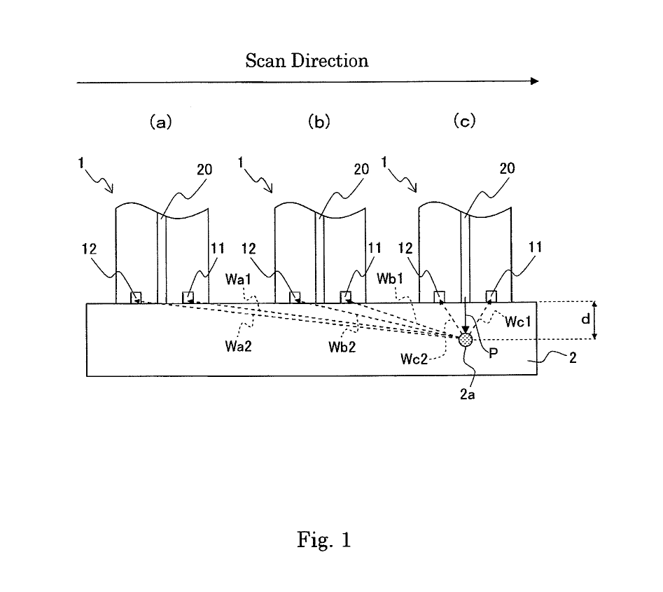

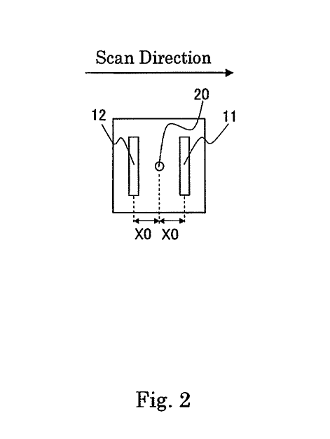

FIG. 1 is a cross sectional view of a photoacoustic wave measurement instrument 1 according to a first embodiment of the present invention. FIG. 2 is a plan view of the photoacoustic wave measurement instrument 1 according to the first embodiment of the present invention. The photoacoustic wave measurement instrument 1 includes photoacoustic wave detection units 11 and 12, and an optical fiber (light output unit) 20. The photoacoustic wave measurement instrument 1 is in contact with a measurement object 2, and is scanned on the measurement object 2 from left to right, for example.

FIG. 1(a) shows the photoacoustic wave measurement instrument 1 positioned far from blood 2a. When the photoacoustic wave measurement instrument 1 shown in FIG. 1(a) is scanned to right, the photoacoustic wave measurement instrument 1 is positioned slightly far from the blood 2a as shown in FIG. 1(b). When the photoacoustic wave measurement instrument 1 shown in FIG. 1(b) is scanned to right, the photoacoustic wave measurement instrument 1 is positioned directly above the blood 2a as shown in FIG. 1(c).

The optical fiber (light output unit) 20 outputs light (such as pulse light P, but continuous light is conceivable). It should be noted that the optical fiber 20 is connected to a pulse light source (not shown) external to the photoacoustic wave measurement instrument 1. The optical fiber 20 passes through the photoacoustic wave measurement instrument 1. Moreover, the pulse light P output from the optical fiber 20 is shown only in FIG. 1(c) for the sake of illustration.

The measurement object 2 is the finger cushion of the human, for example. The blood 2a in a blood vessel exists in the measurement object 2, when the blood 2a in the blood vessel receives the pulse light P, the blood 2a generates photoacoustic waves Wa1 and Wa2 (refer to FIG. 1(a)), photoacoustic waves Wb1 and Wb2 (refer to FIG. 1(b)), and photoacoustic waves Wc1 and Wc2 (refer to FIG. 1(c)).

The photoacoustic wave detection units 11 and 12 receive the photoacoustic waves Wa1, Wa2, Wb1, Wb2, Wc1, and Wc2, and coverts them into electric signals (such as voltages). It is assumed that the photoacoustic wave detection units 11 and 12 are plural. For example, as shown in FIGS. 1 and 2, the number of photoacoustic wave detection units 11 and 12 is two.

Each of the photoacoustic wave detection units 11 and 12 includes a backing material, a piezoelectric element, electrodes, and a spacer which are not shown, and well known. The spacer is in contact with the measurement object 2, the electrodes are placed on the spacer, the piezoelectric element is placed on the electrodes, and the backing material is placed on the piezoelectric element. The photoacoustic waves Wa1, Wa2, Wb1, Wb2, Wc1, and Wc2 are converted into the electric signals (such as voltages) by the piezoelectric element, and extracted to the outside via the electrodes.

Referring to FIG. 2, it should be noted that both the photoacoustic wave detection units 11 and 12 are separated from the optical fiber 20 in a scan direction by a distance X0.

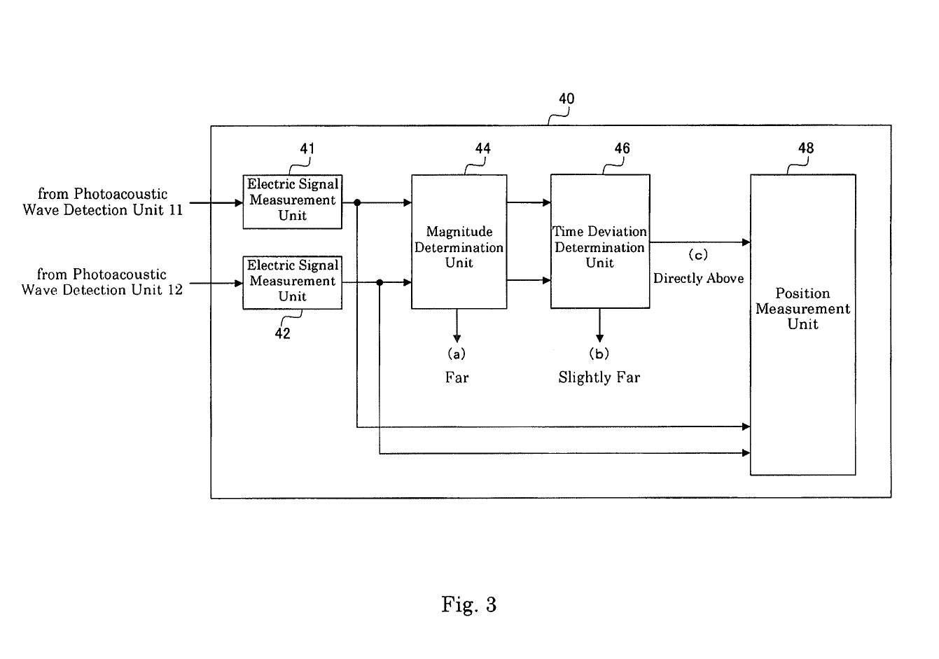

FIG. 3 is a functional block diagram showing a configuration of the photoacoustic wave measurement device 40 according to the first embodiment of the present invention. The photoacoustic wave measurement device 40 includes electric signal measurement units 41 and 42, a magnitude determination unit 44, a time deviation determination unit 46, and a position measurement unit 48. The photoacoustic wave measurement device 40 receives the electric signals from the photoacoustic wave detection units 11 and 12 of the photoacoustic wave measurement instrument 1.

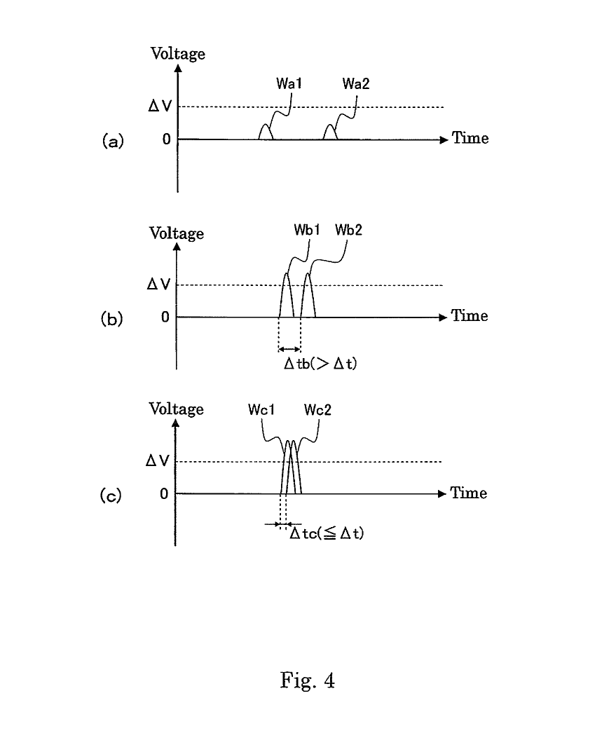

The electric signal measurement unit 41 receives the electric signal from the photoacoustic wave detection unit 11, and outputs a measurement result (such as relationships between the time and the voltage) thereof (refer to Wa1, Wb1, and Wc1 in FIG. 4). The electric signal measurement unit 42 receives the electric signal from the photoacoustic wave detection unit 12, and outputs a measurement result (such as relationships between the time and the voltage) thereof (refer to Wa2, Wb2, and Wc2 in FIG. 4).

The magnitude determination unit 44 receives the measurement results of the electric signals output respectively from the photoacoustic wave detection units 11 and 12 from the electric signal measurement units 41 and 42. Then, the magnitude determination unit 44 determines a magnitude relationship between the magnitude of the electric signal output from each of the photoacoustic wave detection units 11 and 12 and a predetermined threshold ΔV based on the measurement result received from each of the electric signal measurement units 41 and 42.

For example, the magnitude determination unit 44 determines whether both the magnitudes of the electric signals output from the respective photoacoustic wave detection units 11 and 12 are more than (or equal to or more than) the predetermined magnitude threshold ΔV or not.

On this occasion, if the magnitude determination unit 44 determines that both the magnitudes of the electric signals output from the respective photoacoustic wave detection units 11 and 12 are more than the predetermined magnitude threshold ΔV, the magnitude determination unit 44 provides the time deviation determination unit 46 with the measurement results received from the electric signal measurement units 41 and 42.

On the other hand, if the magnitude determination unit 44 determines that at least one of the magnitudes of the electric signals output from the respective photoacoustic wave detection units 11 and 12 is equal to or less than the predetermined magnitude threshold ΔV (refer to FIG. 4(a)), the magnitude determination unit 44 does not provide the time deviation determination unit 46 with the measurement results received from the electric signal measurement units 41 and 42. In this case, the magnitude determination unit 44 may output such a determination result that the photoacoustic wave measurement instrument 1 is positioned far from the blood 2a (refer to FIG. 1(a)).

The time deviation determination unit 46 receives the measurement results via the magnitude determination unit 44 from the electric signal measurement units 41 and 42. Then, the time deviation determination unit 46 determines whether a deviation in time between the electric signals output from the respective photoacoustic wave detection units 11 and 12 is in a predetermined range (equal to or more than 0, and equal to or less than a predetermined time threshold Δt, for example) or not based on the measurement results received from the electric signal measurement units 41 and 42.

For example, the time deviation determination unit 46 determines whether a deviation in time between rising time points of the electric signals output from the respective photoacoustic wave detection units 11 and 12 is equal to or more than 0, and equal to or less than the predetermined time threshold Δt (or equal to or more than 0 and less than Δt) or not.

On this occasion, if the time deviation determination unit 46 determines that a deviation Δtc in time between the rising time points of the electric signals output from the respective photoacoustic wave detection units 11 and 12 is equal to or more than 0, and equal to or less than the predetermined time threshold Δt (refer to FIG. 4(c)), the time deviation determination unit 46 outputs such a determination result that the photoacoustic wave measurement instrument 1 is directly above the blood 2a (refer to FIG. 1(c)) to the position measurement unit 48.

On the other hand, if the time deviation determination unit 46 determines that a deviation Δtb in time between the rising time points of the electric signals output from the respective photoacoustic wave detection units 11 and 12 are more than the predetermined time threshold Δt (refer to FIG. 4(b)), the time deviation determination unit 46 outputs none to the position measurement unit 48. In this case, the time deviation determination unit 46 may output such a determination result that the photoacoustic wave measurement instrument 1 is positioned slightly far from the blood 2a (refer to FIG. 1(b)).

The situation where the time deviation determination unit 46 provides the position measurement unit 48 with such the determination result that the photoacoustic wave measurement instrument 1 is directly above the blood 2a (refer to FIG. 1(c)) means a situation where the magnitude determination unit 44 determines that the magnitudes of the electric signals respectively output from the photoacoustic wave detection units 11 and 12 are more than the threshold ΔV, and simultaneously, the time deviation determination unit 46 determines that the deviation in time between the electric signals respectively output from the photoacoustic wave detection units 11 and 12 is in the predetermined range (equal to or more than 0, and equal to or less than the time threshold Δt).

If the position measurement unit 48 receives such the determination result that the photoacoustic wave measurement instrument 1 is directly above the blood 2a (refer to FIG. 1(c)) from the time deviation determination unit 46, the position measurement unit 48 measures the position of the blood 2a (photoacoustic wave generation part) at which the photoacoustic waves Wc1 and Wc2 are generated in the measurement object 2.

In this case, the position measurement unit 48 measures the position of the blood 2a (photoacoustic wave generation part) while it is assumed that the blood 2a (photoacoustic wave generation part) exists on the extension line of (directly below, for example) the optical fiber (light output unit) 20. The position measurement unit 48 receives the measurement results from the electric signal measurement units 41 and 42, thereby measuring the position of the blood 2a (photoacoustic wave generation part). For example, a depth d of the blood 2a with respect to a surface of the measurement object 2 may be measured. It is assumed that it is found out that a time taken by the photoacoustic wave Wc1 to reach the photoacoustic wave detection unit 11 from the blood 2a and a time taken by the photoacoustic wave Wc2 to reach the photoacoustic wave detection unit 12 from the blood 2a are both T based on the measurement results received from the electric signal measurement units 41 and 42. Then, (T×Vs)²=d²+X0² holds true, where Vs is a velocity of the photoacoustic wave in the measurement object 2. X0 and Vs are known, and the depth d of the blood 2a can thus be obtained.

A description will now be given of an operation of the first embodiment of the present invention.

First, before the description of the operation, the positional relationships between the photoacoustic wave measurement instrument 1 and the blood 2a in FIGS. 1(a), 1(b), and 1(c), and the relationships between the results of the comparison of the electric signal with the magnitude threshold ΔV and the time threshold Δt are shown in Table 1.

TABLE 1

| | | | EQUAL TO OR |
|---|---|---|---|
| | | MORE THAN | LESS THAN TIME |
| | DISTANCE FROM | MAGNITUDE | DEVIATION |
| | BLOOD 2a | THRESHOLD ΔV | THRESHOLD Δt |
| (a) | FAR | x | — |
| (b) | SLIGHTLY FAR | ∘ | x |
| (c) | DIRECTLY | ∘ | ∘ |
| | ABOVE | | |

∘: Condition is satisfied
x: Condition is not satisfied
—: Not determined

When the scan of the photoacoustic wave measurement instrument 1 starts, the photoacoustic wave measurement instrument 1 is positioned far from the blood 2a as shown in FIG. 1(a).

On this occasion, the external pulse light source (not shown) generates the pulse light P, and the pulse light P is output from the optical fiber 20. The pulse light P is fed to the measurement object 2.

The pulse light P reaches the blood 2a in the blood vessel of the measurement object 2. Then, the blood 2a in the blood vessel absorbs the pulse light P, and the dilatational waves (photoacoustic waves Wa1 and Wa2) are output from the blood 2a in the blood vessel.

The photoacoustic waves Wa1 and Wa2 transmit through the measurement object 2, and reach the photoacoustic wave detection units 11 and 12. The photoacoustic wave detection units 11 and 12 respectively convert pressures of the photoacoustic waves Wa1 and Wa2 into the electric signals (such as voltages). The voltages are fed to the electric signal measurement units 41 and 42 of the photoacoustic wave measurement device 40.

FIG. 4 includes charts showing the relationships between the time and the voltage, which are the measurement results by the electric signal measurement units 41 and 42 of the photoacoustic wave measurement device 40 according to the first embodiment, and shows the measurement result of the electric signals obtained from the photoacoustic wave measurement instrument 1 in FIG. 1(a) (FIG. 4(a)), the measurement result of the electric signals obtained from the photoacoustic wave measurement instrument 1 in FIG. 1(b) (FIG. 4(b)), and the measurement result of the electric signals obtained from the photoacoustic wave measurement instrument 1 in FIG. 1(c) (FIG. 4(c)).

(a) when Photoacoustic Wave Measurement Instrument 1 is Far from Blood 2a

As shown in FIG. 1(a), the photoacoustic wave measurement instrument 1 is far from the blood 2a. Thus, the photoacoustic waves Wa1 and Wa2 are weak, and the magnitudes of the electric signals (voltages) obtained from the photoacoustic waves Wa1 and Wa2 are small, and are both equal to or less than the magnitude threshold ΔV (refer to FIG. 4(a)).

In this case, the magnitude determination unit 44 does not feed the measurement results received from the electric signal measurement units 41 and 42 to the time deviation determination unit 46. The magnitude determination unit 44 outputs such the determination result that the photoacoustic wave measurement instrument 1 is positioned far from the blood 2a (refer to FIG. 1(a)).

(b) when Photoacoustic Wave Measurement Instrument 1 is Slightly Far from Blood 2a

When the photoacoustic wave measurement instrument 1 is scanned from the state shown in FIG. 1(a), the photoacoustic wave measurement instrument 1 is positioned slightly far from the blood 2a as shown in FIG. 1(b).

As shown in FIG. 1(b), though the photoacoustic wave measurement instrument 1 is slightly far from the blood 2a, the photoacoustic wave measurement instrument 1 is closer to the blood 2a than the photoacoustic wave measurement instrument 1 in the state shown in FIG. 1(a). Thus, the photoacoustic waves Wb1 and Wb2 are stronger than the photoacoustic waves Wa1 and Wa2, and both the magnitudes of the electric signals (voltages) obtained from the photoacoustic waves Wb1 and Wb2 are more than the magnitude threshold ΔV (refer to FIG. 4(b)).

In this case, the magnitude determination unit 44 feeds the measurement results received from the electric signal measurement units 41 and 42 to the time deviation determination unit 46.

As shown in FIG. 1(b), the photoacoustic wave measurement instrument 1 is slightly far from the blood 2a, and the difference between a distance traveled by the photoacoustic wave Wb1 and a distance traveled by the photoacoustic wave Wb2 is thus not negligible. Thus, a difference between the time taken by the photoacoustic wave Wb1 to reach the photoacoustic wave detection unit 11 and the time taken by the photoacoustic wave Wb2 to reach the photoacoustic wave detection unit 12 is not negligible either. Therefore, the deviation Δtb in time between the rising time points of the electric signals obtained from the photoacoustic waves Wb1 and Wb2 is not negligible (for example, more than the predetermined time threshold Δt) referring to FIG. 4(b).

In this case, the time deviation determination unit 46 does not specifically output anything to the position measurement unit 48. The time deviation determination unit 46 outputs such the determination result that the photoacoustic wave measurement instrument 1 is positioned slightly far from the blood 2a (refer to FIG. 1(b)).

(c) when Photoacoustic Wave Measurement Instrument 1 is Directly Above Blood 2a

When the photoacoustic wave measurement instrument 1 is scanned from the state shown in FIG. 1(b), the photoacoustic wave measurement instrument 1 is positioned directly above the blood 2a as shown in FIG. 1(c).

As shown in FIG. 1(c), the photoacoustic wave measurement instrument 1 is closer to the blood 2a than the photoacoustic wave measurement instrument 1 in the state shown in FIG. 1(a). Thus, the photoacoustic waves Wc1 and Wc2 are stronger than the photoacoustic waves Wa1 and Wa2, and both the magnitudes of the electric signals (voltages) obtained from the photoacoustic waves Wc1 and Wc2 are more than the magnitude threshold ΔV (refer to FIG. 4(c)).

In this case, the magnitude determination unit 44 feeds the measurement results received from the electric signal measurement units 41 and 42 to the time deviation determination unit 46.

As shown in FIG. 1(c), the photoacoustic wave measurement instrument 1 is directly above the blood 2a. Moreover, referring to FIG. 2, the distance between the photoacoustic wave detection unit 11 and the optical fiber 20 and the distance between the photoacoustic wave detection unit 12 and the optical fiber 20 are both X0, and are equal to each other. Thus, the distance traveled by the photoacoustic wave Wc1 and the distance traveled by the photoacoustic wave Wc2 are equal to each other. Thus, the time taken by the photoacoustic wave Wc1 to reach the photoacoustic wave detection unit 11 and the time taken by the photoacoustic wave Wc2 to reach the photoacoustic wave detection unit 12 are equal to each other. Therefore, the deviation Δtc in time between the rising time points of the electric signals obtained from the photoacoustic waves Wc1 and Wc2 is so small as to be negligible (for example, equal to or less than the predetermined time threshold Δt) referring to FIG. 4(c).

In this case, the time deviation determination unit 46 outputs such the determination result that the photoacoustic wave measurement instrument 1 is positioned directly above the blood 2a (refer to FIG. 1(c)) to the position measurement unit 48. The position measurement unit 48 measures the position of the blood 2a (photoacoustic wave generation part) while it is assumed that the blood 2a (photoacoustic wave generation part) exists on the extension line of (directly below, for example) the optical fiber 20. The position measurement unit 48 receives the measurement results from the electric signal measurement units 41 and 42, thereby measuring the position (such as the depth d of the blood 2a) of the blood 2a (photoacoustic wave generation part).

It is possible to determine whether the blood 2a (photoacoustic wave generation part) exists on the extension line of (directly below, for example) the optical fiber 20 of the photoacoustic wave measurement instrument 1 (refer to FIG. 1(c)) or not (refer to FIGS. 1(a) and (b)) according to the first embodiment.

Moreover, the position measurement unit 48 measures the position of the blood 2a while it is assumed that the blood 2a exists on the extension line of the optical fiber 20 of the photoacoustic wave measurement instrument 1 in the photoacoustic wave measurement device 40. On this occasion, the photoacoustic wave measurement device 40 carries out such the measurement when the blood 2a actually exists on the extension line of the optical fiber 20 of the photoacoustic wave measurement instrument 1 (refer to FIG. 1(c)). The position of the blood 2a thus can be precisely measured by the photoacoustic wave measurement instrument 1.

A description is given of the first embodiment while it is assumed that the photoacoustic wave measurement device 40 includes the magnitude determination unit 44. However, such a variation that the photoacoustic wave measurement device 40 does not include the magnitude determination unit 44 is conceivable.

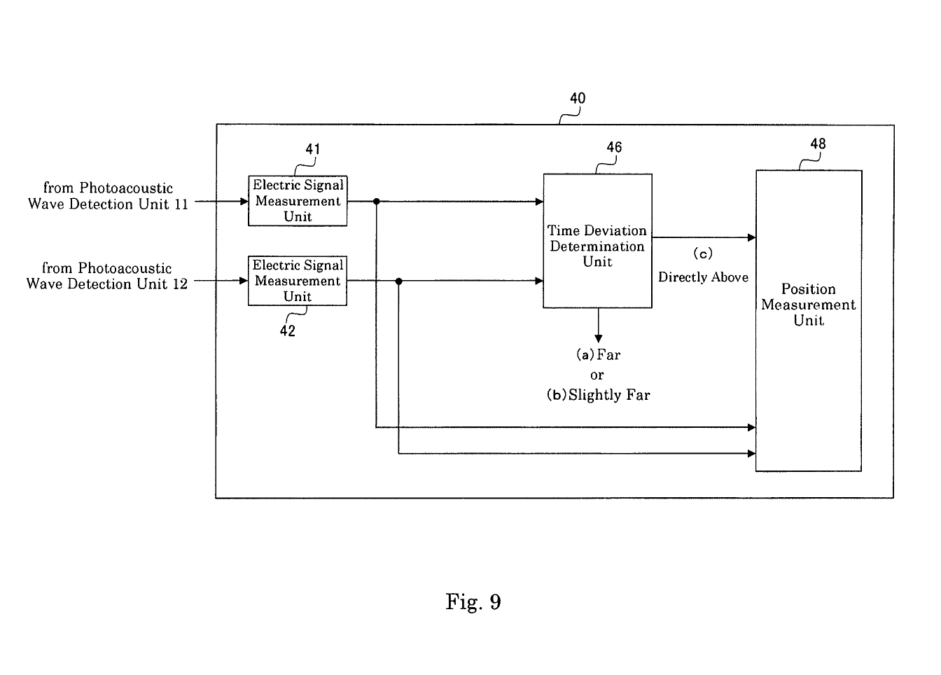

FIG. 9 is a functional block diagram showing a configuration of the photoacoustic wave measurement device 40 according to the variation of the first embodiment of the present invention. The photoacoustic wave measurement device 40 according to the variation of the first embodiment of the present invention includes the electric signal measurement units 41 and 42, the time deviation determination unit 46, and the position measurement unit 48.

The electric signal measurement units 41 and 42 and the position measurement unit 48 are the same as those of the first embodiment (refer to FIG. 3), and hence a description thereof is omitted.

The time deviation determination unit 46 directly (not via the magnitude determination unit 44) receives the measurement results from the electric signal measurement units 41 and 42. The determination method by the time deviation determination unit 46 is the same as that of the first embodiment, and a description thereof, therefore, is omitted.

However, if the time deviation determination unit 46 determines that the time deviation Δtb in time between the rising time points of the electric signals respectively output from the photoacoustic wave detection units 11 and 12 is more than the predetermined time threshold Δt, the time deviation determination unit 46 outputs such a determination result that the photoacoustic wave measurement instrument 1 is positioned far from (refer to FIG. 1(a)), or slightly far from (refer to FIG. 1(b)) the blood 2a. If the photoacoustic wave measurement instrument 1 is positioned slightly far from the blood 2a (refer to FIG. 1(b)), Δtb is more than Δt, and if the photoacoustic wave measurement instrument 1 is positioned far from the blood 2a (refer to FIG. 2(a)), Δtb further increases, and Δtb further exceeds Δt.

If the photoacoustic wave measurement instrument 1 is positioned far from the blood 2a (refer to FIG. 1(a)), the photoacoustic waves Wa1 and Wa2 are weak. However, if the electric signal measurement units 41 and 42 can carry out precise measurement high in S/N ratio, even if the photoacoustic wave measurement instrument 1 is positioned far from the blood 2a, the deviation in time between the rising time points of the electric signals respectively output from the photoacoustic wave detection units 11 and 12 can be measured, and the time deviation determination unit 46 can makes the determination.

The photoacoustic wave measurement device 40 according to the variation of the first embodiment can provide the same effect as of the first embodiment. It should be noted that a measurement by such a variation that the photoacoustic wave measurement device 40 does not include the magnitude determination unit 44 can be made in other embodiments.

### Second Embodiment

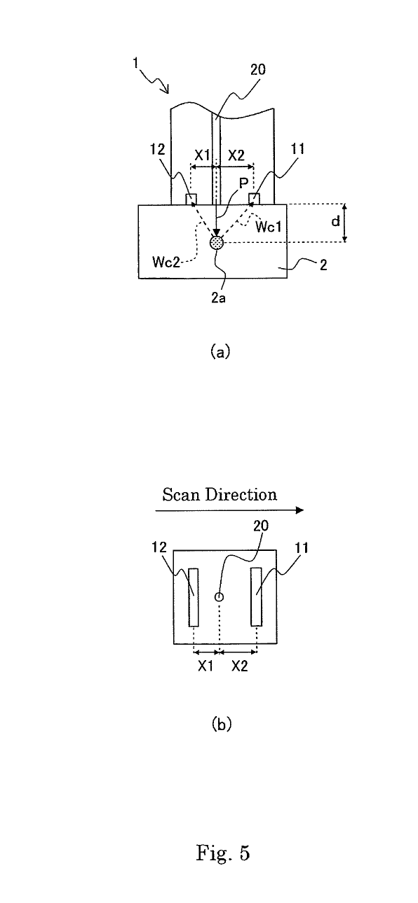

A second embodiment is different from the first embodiment in such a point that the distance between the photoacoustic wave detection unit 11 and the optical fiber 20 and the distance between the photoacoustic wave detection unit 12 and the optical fiber 20 are different from each other (refer to FIG. 5(b)).

FIG. 5 includes a cross sectional view (FIG. 5(a)) and a plan view (FIG. 5(b)) of the photoacoustic wave measurement instrument 1 according to the second embodiment of the present invention. The photoacoustic wave measurement instrument 1 includes the photoacoustic wave detection units 11 and 12, and the optical fiber (light output unit) 20. Hereinafter, like components are denoted by like numerals as of the first embodiment of the photoacoustic wave measurement instrument 1, and will be described in no more details.

The optical fiber (light output unit) 20 is the same as that of the first embodiment, and a description thereof, therefore, is omitted. The photoacoustic wave detection units 11 and 12 are also the same as those of the first embodiment. It should be noted that the positions of the photoacoustic wave detection units 11 and 12 are different from those of the first embodiment.

In other words, the photoacoustic wave detection unit 11 is separated from the optical fiber 20 in the scanning direction by a distance X2. The photoacoustic wave detection unit 12 is separated from the optical fiber 20 in the scanning direction by a distance X1. It should be noted that X1 and X2 are different from each other.

FIG. 5(a) shows such a state that the optical fiber 20 of the photoacoustic wave measurement instrument 1 is directly above the blood 2a. Reference numeral d denotes the depth of the blood 2a with respect to the surface of the measurement object 2.

The distance from the blood 2a to the photoacoustic wave detection unit 11 is the square root of d²+X2². The distance from the blood 2a to the photoacoustic wave detection unit 12 is the square root of d²+X1². Then, a deviation Δt0 between a time taken by the photoacoustic wave Wc1 to reach the photoacoustic wave detection unit 11 from the blood 2a and a time taken by the photoacoustic wave Wc2 to reach the photoacoustic wave detection unit 12 from the blood 2a is represented as (square root of ((d²+X2²)−square root of (d²+X1²))/Vs) where the velocity of the photoacoustic wave in the measurement object 2 is Vs. If Δt0 is obtained according to the above-mentioned equation, the depth d of the blood 2a has a deviation to a certain degree, and it is thus conceivable to use an approximate representative value. Moreover, if X1 and X2 are fairly larger than d, it is conceivable to neglect d, and to consider (X2−X1)/Vs as Δt0.

The deviation between the time taken by the photoacoustic wave Wc1 to reach the photoacoustic wave detection unit 11 from the blood 2a and the time taken by the photoacoustic wave Wc2 to reach the photoacoustic wave detection unit 12 from the blood 2a appears as a deviation in time of the electric signals respectively output from the photoacoustic wave detection units 11 and 12.

In other words, Δt0 is a deviation in time between the electric signals respectively output from the photoacoustic wave detection units 11 and 12 if it is assumed that the blood 2a (photoacoustic wave generation part) exists on the extension line of the optical fiber 20.

The photoacoustic wave measurement device 40 according to the second embodiment of the present invention includes the electric signal measurement units 41 and 42, the magnitude determination unit 44, the time deviation determination unit 46, and the position measurement unit 48. The configuration of the photoacoustic wave measurement device 40 according to the second embodiment of the present invention is the same as that of the first embodiment (refer to FIG. 3), and hence description thereof is omitted. Hereinafter, like components are denoted by like numerals as of the first embodiment of the photoacoustic wave measurement device 40, and will be described in no more details.

The electric signal measurement units 41 and 42 and the magnitude determination unit 44 are the same as those of the first embodiment, and a description thereof, therefore, is omitted,

The time deviation determination unit 46 determines whether the deviation in time between the electric signals output from the respective photoacoustic wave detection units 11 and 12 is in a predetermined range (for example equal to more than (Δt0−Δt) and equal to or less than (Δt0+Δt) where the predetermined range is Δt) or not based on the measurement results received from the electric signal measurement units 41 and 42. Δt0 is in the predetermined range. In other words, the predetermined range includes Δt0. A relationship Δt0−Δt>0 may hold true. In other words, the predetermined range may not include 0.

For example, the time deviation determination unit 46 determines whether a deviation in time between the rising points of the electric signals respectively output from the photoacoustic wave detection units 11 and 12 is equal to or more than (Δt0−Δt) and equal to or less than (Δt0+Δt), (or more than (Δt0−Δt) and less than (Δt0+Δt)) or not.

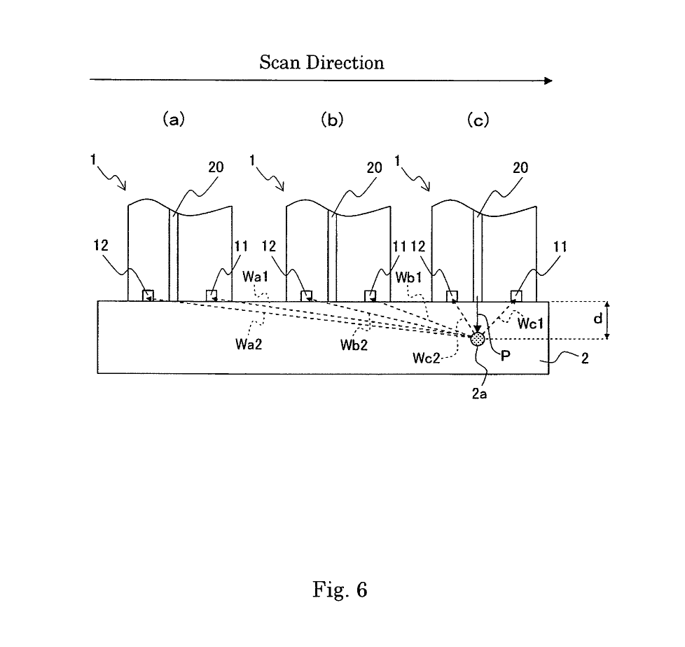

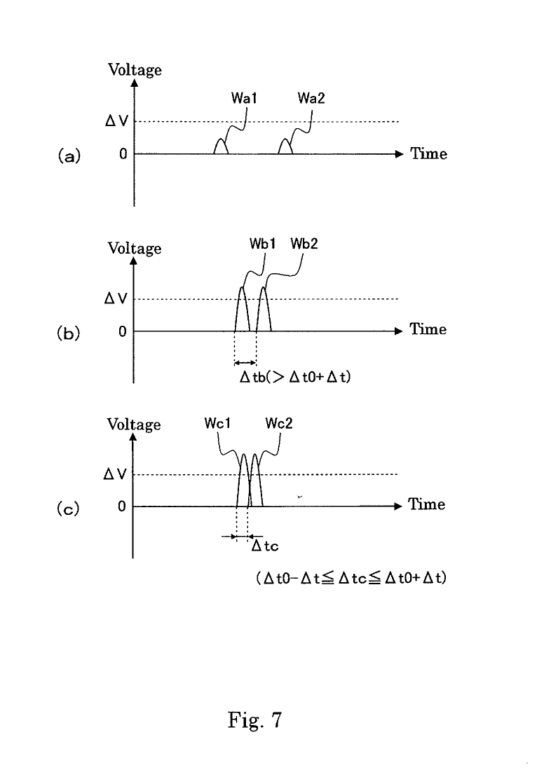

On this occasion, if the time deviation determination unit 46 determines that the deviation Δtc in time between the rising time points of the electric signals output from the respective photoacoustic wave detection units 11 and 12 is equal to or more than (Δt0−Δt), and equal to or less than (Δt0+Δt) (refer to FIG. 7(c)), the time deviation determination unit 46 outputs such a determination result (refer to FIG. 6(c)) that the optical fiber 20 of the photoacoustic wave measurement instrument 1 is directly above the blood 2a to the position measurement unit 48. If the optical fiber 20 of the photoacoustic wave measurement instrument 1 is positioned directly above the blood 2a, though a relationship Δt=Δt0 ideally holds true, if a relationship Δt0−Δt≦Δtc≦Δt0+Δt holds true considering a measurement error and a variation in depth d of the blood 2a, it is assumed to make such a determination that the optical fiber 20 of the photoacoustic wave measurement instrument 1 is directly above the blood 2a.

On the other hand, if the time deviation determination unit 46 determines that the deviation Δtb in time between the rising time points of the electric signals output from the respective photoacoustic wave detection units 11 and 12 is less than (Δt0−Δt) or more than (Δt0+Δt) (refer to FIG. 7(b)), the time deviation determination unit 46 outputs none to the position measurement unit 48. In this case, the time deviation determination unit 46 may output such a determination result that the photoacoustic wave measurement instrument 1 is positioned slightly far from the blood 2a (refer to FIG. 6(b)).

The situation where the time deviation determination unit 46 provides the position measurement unit 48 with such the determination result that the optical fiber 20 of the photoacoustic wave measurement instrument 1 is directly above the blood 2a (refer to FIG. 6(c)) means a situation where the magnitude determination unit 44 determines that the magnitudes of the electric signals respectively output from the photoacoustic wave detection units 11 and 12 are more than the threshold ΔV, and simultaneously, the time deviation determination unit 46 determines that the deviation in time between the electric signals respectively output from the photoacoustic wave detection units 11 and 12 is in the predetermined range (equal to or more than (Δt0−Δt), and equal to or less than (Δt0+Δt)).

If the position measurement unit 48 receives such the determination result that the optical fiber 20 of the photoacoustic wave measurement instrument 1 is directly above the blood 2a (refer to FIG. 6(c)) from the time deviation determination unit 46, the position measurement unit 48 measures the position of the blood 2a (photoacoustic wave generation part) at which the photoacoustic waves Wc1 and Wc2 are generated in the measurement object 2.

In this case, the position measurement unit 48 measures the position of the blood 2a (photoacoustic wave generation part) while it is assumed that the blood 2a (photoacoustic wave generation part) exists on the extension line of (directly below, for example) the optical fiber (light output unit) 20. The position measurement unit 48 receives the measurement results from the electric signal measurement units 41 and 42, thereby measuring the position of the blood 2a (photoacoustic wave generation part). For example, the depth d of the blood 2a with respect to the surface of the measurement object 2 may be measured. It is assumed that the time taken by the photoacoustic wave Wc1 (Wc2) to reach the photoacoustic wave detection unit 11 (12) from the blood 2a is T1 (T2) based on the measurement result received from the electric signal measurement unit 41 (42). Then, (T1×Vs)²=d²+X2² ((T2×Vs)²=d²+X1²) holds true, where Vs is the velocity of the photoacoustic wave in the measurement object 2. X2 (X1) and Vs are known, and the depth d of the blood 2a can thus be obtained.

A description will now be given of an operation of the second embodiment of the present invention.

FIG. 6 is a cross sectional view of the photoacoustic wave measurement instrument 1 while the photoacoustic wave measurement instrument 1 according to the second embodiment of the present invention is scanned along the measurement object 2.

When the scan of the photoacoustic wave measurement instrument 1 starts, the photoacoustic wave measurement instrument 1 is positioned far from the blood 2a as shown in FIG. 6(a).

On this occasion, the external pulse light source (not shown) generates the pulse light P, and the pulse light P is output from the optical fiber 20. The pulse light P is fed to the measurement object 2.

The pulse light P reaches the blood 2a in the blood vessel of the measurement object 2. Then, the blood 2a in the blood vessel absorbs the pulse light P, and the dilatational waves (photoacoustic waves Wa1 and Wa2) are output from the blood 2a in the blood vessel.

The photoacoustic waves Wa1 and Wa2 transmit through the measurement object 2, and reach the photoacoustic wave detection units 11 and 12. The photoacoustic wave detection units 11 and 12 convert pressures of the photoacoustic waves Wa1 and Wa2 into the electric signals (such as voltages). The voltages are fed to the electric signal measurement units 41 and 42 of the photoacoustic wave measurement device 40.

FIG. 7 includes charts showing relationships between the time and the voltage, which are the measurement results by the electric signal measurement units 41 and 42 of the photoacoustic wave measurement device 40 according to the second embodiment, and shows the measurement result of the electric signals obtained from the photoacoustic wave measurement instrument 1 in FIG. 6(a) (FIG. 7(a)), the measurement result of the electric signals obtained from the photoacoustic wave measurement instrument 1 in FIG. 6(b) (FIG. 7(b)), and the measurement result of the electric signals obtained from the photoacoustic wave measurement instrument 1 in FIG. 6(c) (FIG. 7(c)).

(a) when Photoacoustic Wave Measurement Instrument 1 is Far from Blood 2a

As shown in FIG. 6(a), the photoacoustic wave measurement instrument 1 is far from the blood 2a. Thus, the photoacoustic waves Wa1 and Wa2 are weak, and the magnitudes of the electric signals (voltages) obtained from the photoacoustic waves Wa1 and Wa2 are small, and are both equal to or less than the magnitude threshold ΔV (refer to FIG. 7(a)).

In this case, the magnitude determination unit 44 does not feed the measurement results received from the electric signal measurement units 41 and 42 to the time deviation determination unit 46. The magnitude determination unit 44 outputs such the determination result that the photoacoustic wave measurement instrument 1 is positioned far from the blood 2a (refer to FIG. 6(a)).

(b) when Photoacoustic Wave Measurement Instrument 1 is Slightly Far from Blood 2a

When the photoacoustic wave measurement instrument 1 is scanned from the state shown in FIG. 6(a), the photoacoustic wave measurement instrument 1 is positioned slightly far from the blood 2a as shown in FIG. 6(b).

As shown in FIG. 6(b), though the photoacoustic wave measurement instrument 1 is slightly far from the blood 2a, the photoacoustic wave measurement instrument 1 is closer to the blood 2a than the photoacoustic wave measurement instrument 1 in the state shown in FIG. 6(a). Thus, the photoacoustic waves Wb1 and Wb2 are stronger than the photoacoustic waves Wa1 and Wa2, and both the magnitudes of the electric signals (voltages) obtained from the photoacoustic waves Wb1 and Wb2 are more than the magnitude threshold ΔV (refer to FIG. 7(b)).

In this case, the magnitude determination unit 44 feeds the measurement results received from the electric signal measurement units 41 and 42 to the time deviation determination unit 46.

As shown in FIG. 7(b), the photoacoustic wave measurement instrument 1 is slightly far from the blood 2a, the difference between a distance traveled by the photoacoustic wave Wb1 and a distance traveled the photoacoustic wave Wb2 is not negligible. Thus, a difference between the time taken by the photoacoustic wave Wb1 to reach the photoacoustic wave detection unit 11 and the time taken by the photoacoustic wave Wb2 to reach the photoacoustic wave detection unit 12 is not negligible either. Therefore, the deviation Δtb in time between the rising time points of the electric signals obtained from the photoacoustic waves Wb1 and Wb2 is not negligible (for example, is more than the (Δt0+Δt)) referring to FIG. 4(b).

In this case, the time deviation determination unit 46 does not specifically output anything to the position measurement unit 48. The time deviation determination unit 46 outputs such the determination result that the photoacoustic wave measurement instrument 1 is positioned slightly far from the blood 2a (refer to FIG. 6(b)).

(c) when Optical Fiber 20 of Photoacoustic Wave Measurement Instrument 1 is Directly Above Blood 2a

When the photoacoustic wave measurement instrument 1 is scanned from the state shown in FIG. 6(b), the optical fiber 20 of the photoacoustic wave measurement instrument 1 is positioned directly above the blood 2a as shown in FIG. 6(c).

As shown in FIG. 6(c), the photoacoustic wave measurement instrument 1 is closer to the blood 2a than the photoacoustic wave measurement instrument 1 in the state shown in FIG. 6(a). Thus, the photoacoustic waves Wc1 and Wc2 are stronger than the photoacoustic waves Wa1 and Wa1, and both the magnitudes of the electric signals (voltages) obtained from the photoacoustic waves Wc1 and Wc2 are more than the magnitude threshold ΔV (refer to FIG. 7(c)).

In this case, the magnitude determination unit 44 feeds the measurement results received from the electric signal measurement units 41 and 42 to the time deviation determination unit 46.

As shown in FIG. 6(c), the optical fiber 20 of the photoacoustic wave measurement instrument 1 is directly above the blood 2a. On this occasion, the distance between the photoacoustic wave detection unit 11 and the optical fiber 20 is X2, the distance between the photoacoustic wave detection unit 12 and the optical fiber 20 is X1, and X1 and X2 are different from each other referring to FIG. 5. Thus, the distance traveled by the photoacoustic wave Wc1 and the distance traveled by the photoacoustic wave Wc2 are different from each other. Thus, the time taken by the photoacoustic wave Wc1 to reach the photoacoustic wave detection unit 11 and the time taken by the photoacoustic wave Wc2 to reach the photoacoustic wave detection unit 12 are different from each other. The deviation in time between them is Δt0 as described before.

Therefore, the deviation Δtc in time between the rising time points of the electric signals obtained from the photoacoustic waves Wc1 and Wc2 is approximately equal to Δt0 (for example, Δt0−Δt≦Δtc≦Δt0+Δt) referring to FIG. 7(c).

In this case, the time deviation determination unit 46 outputs such the determination result that the optical fiber 20 of the photoacoustic wave measurement instrument 1 is positioned directly above the blood 2a (refer to FIG. 7(c)) to the position measurement unit 48. The position measurement unit 48 measures the position of the blood 2a (photoacoustic wave generation part) while it is assumed that the blood 2a (photoacoustic wave generation part) exists on the extension line of (directly below, for example) the optical fiber 20. The position measurement unit 48 receives the measurement results from the electric signal measurement units 41 and 42, thereby measuring the position (such as the depth d of the blood 2a) of the blood 2a (photoacoustic wave generation part).

It is possible to determine whether the blood 2a (photoacoustic wave generation part) exists on the extension line of (directly below, for example) the optical fiber 20 of the photoacoustic wave measurement instrument 1 (refer to FIG. 7(c)) or not (refer to FIGS. 7(a) and (b)) according to the second embodiment.

Moreover, the position measurement unit 48 measures the position of the blood 2a while it is assumed that the blood 2a exists on the extension line of the optical fiber 20 of the photoacoustic wave measurement instrument 1 in the photoacoustic wave measurement device 40. On this occasion, the photoacoustic wave measurement device 40 carries out this measurement when the blood 2a actually exists on the extension line of the optical fiber 20 of the photoacoustic wave measurement instrument 1 (refer to FIG. 6(c)). The position of the blood 2a thus can be precisely measured by the photoacoustic wave measurement instrument 1.

A description is given of the embodiments assuming that the photoacoustic wave measurement instrument 1 includes two photoacoustic wave detection units 11 and 12. However, the photoacoustic wave measurement instrument 1 may include at least three photoacoustic wave detection units.

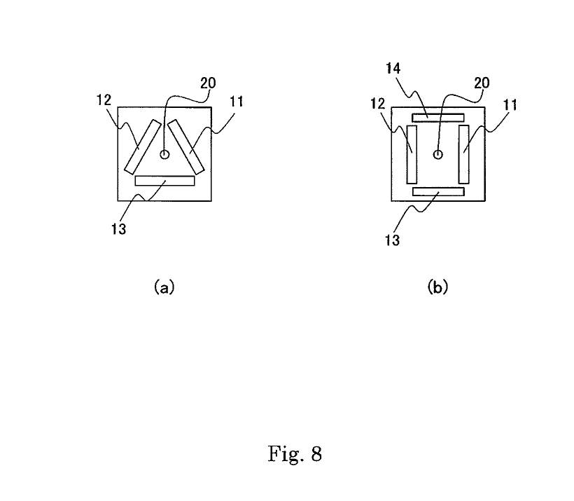

FIG. 8 includes a plan view of the photoacoustic wave measurement instrument 1 including three photoacoustic wave detection units (FIG. 8(a)), and a plan view of the photoacoustic wave measurement instrument 1 including four photoacoustic wave detection units (FIG. 8(b)).

There is provided such a configuration that the three photoacoustic wave detection units 11, 12, and 13 or the four photoacoustic wave detection units 11, 12, 13, and 14 enclose the optical fiber 20 as shown in FIGS. 8(a) and 8(b). Even if such the photoacoustic wave measurement instrument 1 is used, the position of the blood 2a can be precisely measured by the photoacoustic wave measurement instrument 1.

Moreover, the above-described embodiment may be realized in the following manner. A computer is provided with a CPU, a hard disk, and a media (such as a floppy disk (registered trade mark) and a CD-ROM) reader, and the media reader is caused to read a medium recording a program realizing the above-described respective components such as the photoacoustic wave measurement device 40, thereby installing the program on the hard disk. This method may also realize the above-described functions.

What is claimed is:

1. A photoacoustic wave measurement device for receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, comprising:
a time deviation determination unit that determines whether time deviations among the electric signals output from the respective photoacoustic wave detection units are in a predetermined range; and
a position measurement unit that measures a position of a photoacoustic wave generation part of the measurement object which generates the photoacoustic wave if the time deviation determination unit determines that the time deviations among the electric signals are in the predetermined range.

2. The photoacoustic wave measurement device according to claim 1, wherein the position measurement unit measures the position of the photoacoustic wave generation part while it is assumed that the photoacoustic wave generation part exists on an extension line of the light output unit.

3. The photoacoustic wave measurement device according to claim 1, wherein the predetermined range is equal to or more than 0, and equal to or less than a predetermined time threshold.

4. The photoacoustic wave measurement device according to claim 1, wherein the predetermined range includes a time deviation of the electric signal output from each of the photoacoustic wave detection units while it is assumed that the photoacoustic wave generation part exists on an extension line of the light output unit.

5. The photoacoustic wave measurement device according to claim 4, wherein the predetermined range does not include 0.

6. The photoacoustic wave measurement device according to claim 1, comprising a magnitude determination unit that determines a magnitude relationship between a magnitude of the electric signal output from each of the photoacoustic wave detection units and a predetermined magnitude threshold, wherein the position measurement unit measures the position of the photoacoustic wave generation part of the measurement object which generates the photoacoustic wave if the magnitude determination unit determines that the magnitudes of the electric signals are more than the magnitude threshold, and the time deviation determination unit determines that the time deviations among the electric signals are in the predetermined range.

7. A photoacoustic wave measurement method of measuring a photoacoustic wave by receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, comprising:
a time deviation determination step that determines whether time deviations among the electric signals output from the respective photoacoustic wave detection units are in a predetermined range; and
a position measurement step that measures a position of a photoacoustic wave generation part of the measurement object which generates the photoacoustic wave if the time deviation determination step determines that the time deviations among the electric signals are in the predetermined range.

8. A computer-readable medium having a program of instructions for execution by a computer to perform a photoacoustic wave measurement process of measuring a photoacoustic wave by receiving electric signals from a photoacoustic wave measurement instrument including a light output unit for outputting light, and a plurality of photoacoustic wave detection units each for receiving a photoacoustic wave generated by the light in a measurement object, and converting the photoacoustic wave into the electric signal, said process comprising:
a time deviation determination step that determines whether time deviations among the electric signals output from the respective photoacoustic wave detection units are in a predetermined range; and
a position measurement step that measures a position of a photoacoustic wave generation part of the measurement object which generates the photoacoustic wave if the time deviation determination step determines that the time deviations among the electric signals are in the predetermined range.
